(19) United States
(12) Patent Application Publication
(10) Pub. No.: US 2026/0008738 A1
(43) Pub. Date: Jan. 8, 2026

(54) HYDROFLUOROETHER COMPOSITION AND METHOD FOR ITS PREPARATION

(71) Applicant: SOLVAY SPECIALTY POLYMERS ITALY S.P.A., Bollate (Milano) (IT)

(72) Inventors: Emanuela Antenucci, Saronno (IT); Letanzio Bragante, DUE CARRARE (PD) (IT); Manuel Gregori, Rovellasca (IT); Valeriy Kapelyushko, Alessandria (IT); Stefano Millefanti, Tradate (IT)

(73) Assignee: SOLVAY SPECIALTY POLYMERS ITALY S.P.A., Bollate (Milano) (IT)

(21) Appl. No.: 18/880,215

(22) PCT Filed: Jun. 22, 2023
(86) PCT No.: PCT/EP2023/066983

§ 371 Date: Dec. 30, 2024

(30) Foreign Application Priority Data
Jun. 29, 2022 (EP) 22181823.0

(51) Int. Cl. C07C 41/42; C07C 43/12

(52) U.S. Cl. CPC C07C 41/42; C07C 43/126

(57) ABSTRACT

A liquid composition comprising one or more compound of formula (I): CFHX—CF₂—O—R—O—CF₂—CFHX (I), one or more compound of formula (II): CFHX—CF₂—O—R—OH (II) wherein: —R in formulas (I) and (II) is independently selected from C2-C10 divalent linear or branched alkyl, optionally including a cycle, an aromatic ring and/or oxygen heteroatoms engaged in ether bonds, X is selected from halogens, H, Rf, wherein Rf is selected from: —a C1-C8 fully or partially fluorinated linear or branched alkyl optionally including a cycle, or—a C1-C3 fully or partially fluorinated alkoxy, the two instances of X in formula (I) being the same or different, preferably being the same, —the amount of the one or more compounds of formula (II) is from 0.002 to 5% by weight of the one or more compounds of formula (I).

### TECHNICAL FIELD

The invention relates to a hydrofluoroether composition which can find application as solvent. The invention also relates to a method of preparation of said composition.

This application claims priority from the European patent application filed on 29 Jun. 2022 in EUROPE with Nr. 22181823.0, the whole content of this application being incorporated herein by reference for all purposes.

### BACKGROUND ART

Various methods for producing hydrofluoroethers from bi-functional alcohols are known in the art. U.S. Pat. No. 4,208,081 (Du Pont) describes the preparation of hydrofluoroethers using ethylene glycol in a diethyl ether solution and tetrafluoroethylene (TFE). However the reaction is not complete as two reaction products are formed in almost equal amounts, one where both-OH groups of the ethylene glycol are converted to ether groups, and another where only one of the —OH groups of the ethylene glycol has been converted to ether, while the other remains as a free hydroxyl group.

RU1810324 to Natalya Guseva reports the preparation of hydrofluoroethers from a reaction between ethylene glycol and TFE in diglyme solvent in a anhydrous process with a 78% yield.

Solvents based on hydrofluoroethers are very advantageous if compared to alternative materials which are available in the market because they have low GWP (Global warming potential) low flammability and are thus safe and easy to handle. However hydrofluoroethers have in general a relatively low polarity so that this limits somehow their range of applicability. On the other hand mixing hydrofluoroethers with other more polar solvents may cause the composition to be incompatible with certain materials. Therefore there is a need for solvent compositions having all the advantages of hydrofluoroether based solvents, including the same material compatibility, in combination with a broader range of applicability in terms of what these solvent compositions are able to solubilize. The composition of the present invention maintain intact all the properties of hydrofluoroether based solvents while having a finely tuned increased polarity so it has a broader range of applicability as solvent than the pure materials and at the same time preserving all the advantages of using hydrofluoroether based solvents.

Also there is still a need for a composition which can be easily prepared using a processes which is simple and has a high yield. The present invention also relates to such a process.

### SUMMARY OF THE INVENTION

The present invention relates to a liquid composition comprising one or more compound of formula (I):

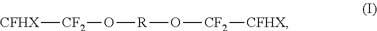

one or more compound of formula (II):

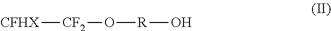

wherein:
   R in formulas (I) and (II) is independently selected from C2-C10 divalent linear or branched alkyl, optionally including a cycle, an aromatic ring and/or oxygen heteroatoms engaged in ether bonds,
   X is selected from halogens, H, Rf, wherein Rf is selected from:
      a C1-C8 fully or partially fluorinated linear or branched alkyl optionally including a cycle, or
      a C1-C3 fully or partially fluorinated alkoxy,
the two instances of X in formula (I) being the same or different, preferably being the same,
   the amount of the one or more compounds of formula (II) is from 0.002 to 5% by weight of the one or more compounds of formula (I).

### DETAILED DESCRIPTION OF THE INVENTION

It is thus an object of the present invention to provide a liquid composition comprising one or more compounds according to formula (I):

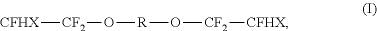

and one or more compounds according to formula (II):

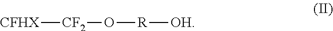

In the composition of the invention the total amount of said one or more compounds of formula (II) is from 0.002 to 5%, preferably from 0.005 to 3%, more preferably, from 0.01 to 2%, most preferably from 0.05 to 1% by weight of the total amount of said one or more compounds of formula (I).

In formulas (I) and (II):
R is a C2-C10 divalent linear or branched alkyl, which may include a cycle, an aromatic ring or oxygen (O) heteroatoms engaged in ether bonds. R is preferably C2-C6 divalent linear alkyl not containing aromatic moieties. More preferably R is selected from:

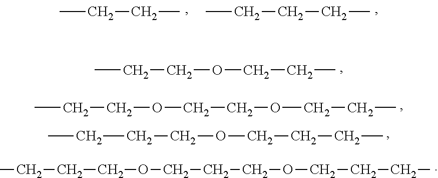

R in the compounds according to formula (I) and (II) can be the same or different and is preferably the same.
X is selected from halogen, H and Rf. In case X is an halogen it is preferably Cl or F. Rf is selected from a C1-C8 fully or partially fluorinated linear or branched alkyl optionally including a cycle, or a C1-C3 fully or partially fluorinated alkoxy.

In case X is Rf, preferably Rf is a C1-C3 fully fluorinated alkyl even more preferably is —CF₃). In a further preferred embodiment X is selected from F, CI and CF₃ and most preferably is F.

In the composition of the invention it is preferred that the total amount of compounds according to formulas (I) and (II) is at least 50%, more preferably at least 70%, even more preferably at least 85%, most preferably at least 90% by weight, based on the total weight of the composition.

In one embodiment the composition of the invention consists essentially of compounds according to formulas (I) and (II). For “consists essentially” it is intended that the total amount of compounds according to formulas (I) and (II) represents at least 95%, preferably at least 97%, most preferably at least 99% by weight based on the total weight of the composition.

A composition according to the invention can be prepared with any method known to the skilled person e.g. mixing the one or more compound according to formula (I) with the one or more compound according to formula (II).

A preferred method for making the composition of the invention is a the reaction between a bifunctional alcohol and a fluorinated olefin in a selected polar aprotic solvent and in the presence of a basic catalyst. This method provides high yields and can be performed easily in mild conditions and without requiring ingredients which are harmful for the environment and it allows to obtain directly a composition according to the invention already possessing the desired balance among the compounds according to formulas (I) and (II) without the need to mix the different components.

As apparent to the skilled person the one or more compounds according to formulas (I) and (II) are hydrofluoroethers. An hydrofluoroether is defined as a chemical compound having the general formula R—O—R′ wherein at least one of R and R′ comprises at least one C—F bond and at least one C—H bond.

One way of forming hydrofluoroethers is to react a chemical compound carrying at least one-OH group which is part of an alcohol or of a phenol group with a fluorinated olefin which can be partially or fully fluorinated. The reaction between the —OH group and the C═C double bond of the olefin can be described as an addition to the C═C double bond where one of its carbon atoms forms a C—O bond and the other a C—H bond.

In a first step (A) of the process of the present invention a mixture is provided comprising one or more polar aprotic organic solvents and one or more bifunctional alcohol.

Suitable polar aprotic organic solvents for use in the process of the present invention are polar aprotic organic solvents having a boiling point measured at atmospheric pressure (1 atm) of from of 60 to 170° C., preferably of from 70° C. to 90° C.

Particularly suitable polar aprotic solvents for use herein are those carrying a nitrile group, a particularly preferred solvent is acetonitrile.

The other essential component of the mixture to be provided in step A of the process of the present invention is a bifunctional alcohol. A bifunctional alcohol is a chemical compound carrying two-OH groups each of which is part of an alcohol group (i.e. it is covalently bonded to an aliphatic carbon atom which is not part of a carbonyl group —C═O).

These chemical compounds typically correspond to the general formula

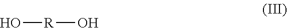

wherein R is defined as above for the formulas (I) and (II).

Examples of bifunctional alcohols suitable for use in the present invention are ethylene glycol, di-ethylene glycol, tri-ethylene glycol, propylene glycol, di-propylene glycol, tri-propylene glycol, 1,3-propan-diol, cyclohexandiol, cyclohexanedimethanol).

When forming an hydrofluoroether from a bifunctional alcohol the yields tend to be lower as the various —OH groups may have different reactivity, especially after one or more of them have already reacted with the fluorinated olefin to form a first ether bond. The method of the invention allows to obtain an excellent yield with bifunctional alcohols.

In the process of the present invention one or more bifunctional alcohol as defined above is provided in a mixture with one or more polar aprotic solvents selected as defined above. Such solvents are generally good solvents for the bifunctional alcohol so that preferably the mixture provided is homogeneous. The relative amount of the selected one or more polar aprotic organic solvents and of the one or more bifunctional alcohols is preferably at least 1:1 by weight, preferably at least 2:1 more preferably at least 3:1, most preferably at least 4:1. Other solvents may be present in the mixture, but preferably the total amount of the one or more bifunctional alcohol and of the one or more selected polar aprotic solvents represents at least 70%, more preferably at least 80%, even more preferably at least 90%, most preferably at least 95% by weight of the mixture.

In step B of the process of the present invention the mixture provided in step A is reacted with one or more fluorinated olefin in the presence of a basic catalyst.

Preferably the fluorinated olefin is selected among the fully halogenated olefins and more preferably from the group consisting of perfluoromethyl vinyl ether, perfluoroethyl vinyl ether, perfluoropropyl vinyl ether, tetrafluoroethylene (TFE), chlorotrifluoroethylene (CTFE), hexafluoropropylene (HFP) and more preferably is TFE.

The fluorinated olefin can be initially loaded in the reaction vessel or can be advantageously continuously fed in the required amount during the reaction.

The one or more fluorinated olefin has the following general formula

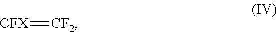

wherein
   X is selected from halogens, H, Rf, wherein Rf is selected from:
      a C1-C8 fully or partially fluorinated linear or branched alkyl optionally including a cycle, or
      a C1-C3 fully or partially fluorinated alkoxy,

The reaction of an —OH carrying compound with a fluorinated olefin can be schematized as follows:

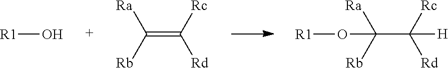

Radical R₁, in accordance with the definition provided above may still contain other —OH groups (e.g. in case of a polyfunctional alcohol). In that case also the resulting hydrofluoroether will still contain the same —OH groups so that it can further react with other molecules of the fluorinated olefin until all of the —OH groups are fully reacted so that their oxygen atoms are all engaged in ether type bonds.

The reaction can be typically performed in a stirred reactor which is preferably sealed. The molar ratio between the —OH groups and the fluorinated olefins is in principle stoichiometric i.e. in order to have complete reaction and not have residual reagents the same molar amount of double bonds from the olefins should be present as the molar amount of —OH groups in the bifunctional alcohols. Naturally in the present case wherein bifunctional alcohols are used each —OH group can react with a different olefin molecule so that for one mole of bifunctional alcohol two moles of fluorinated olefin will be required for a stoichiometric ratio. While a stoichiometric ratio between double bonds and OH groups is ideal, the present invention can be effectively carried out also when one of the components is in a molar excess up to 50%, preferably up to 30%, more preferably up to 20% most preferably up to 10%. In case one component is in excess said component in excess is preferably the fluorinated olefin.

The basic catalyst can be any chemical compound capable of creating a basic environment i.e. to subtract protons from the reagents thus promoting the ionic reaction. Preferred basic catalyst are selected from inorganic hydroxides (such as NaOH, KOH, LiOH, Ca(OH)2, Mg(OH)2), inorganic salts of weak acids (such as alkali metal phosphates or carbonates), organic basic compounds (such as alcolates). Most preferred basic catalysts are NaOH and KOH.

The amount of catalyst to be used is typically from 5% to 100%, preferably 10%-70%, more preferably 15%-50% by moles with respect to the total moles of —OH groups.

Typically the basic catalyst is added to the reactor adding it to the mixture provided in step A under agitation. The reactor is then typically sealed and the fluorinated olefin is pumped in gas form up to a pressure of from 1 to 50 bar, preferably 2-30 bar, more preferably 3-20 bar, most preferably between 4 and 14 bar. In case the fluorinated olefin is in liquid form the olefin can be introduced as a liquid and if it remains in liquid status at the temperature of reaction, the reaction can be carried out at a lower pressure or even at atmospheric pressure.

The reaction typically starts immediately. Preferably, during the reaction, the reactor is maintained at a temperature of from 20° to 90° C., preferably from 30° to 80° C., most preferably from 40° to 70° C. The reaction time can be variable depending on the temperature, pressure and reagents used. Typically the reaction will require from 1 to 20 hours to complete.

After the reaction is completed the reactor is typically vented to remove the excess of fluorinated olefin. At this stage the reactor contains a liquid reacted mixture comprising one or more compounds of formula (I) and one or more compounds of formula (II), in combination with the polar aprotic solvent and with residues of the basic catalyst.

The one or more compounds of formula (I) and one or more compounds of formula (II) can be extracted directly from the reacted mixture with known techniques such as distillation (in optional step C of the present invention). However the reacted mixture obtained in step B still contains dissolved or dispersed solids, typically inorganic solids deriving from the basic catalyst, so that the direct distillation of said reacted mixture would cause the build-up of unwanted solid deposits on the distillation equipment which, while it can be acceptable in lab scale, are more problematic at an industrial scale as it could force the equipment to have frequent stops for cleaning/restoring it. Therefore, preferably, before extracting the hydrofluoroethers via distillation the reacted mixture is purified to remove catalyst residues and solid by-products.

In one embodiment the reacted mixture is purified via an extraction with water. In another embodiment the reacted mixture is purified trough evaporation and re-condensation. These two embodiments will be described in detail below.

### Evaporation/Recondensation Method:

In the optional step D of the present invention the liquid reacted mixture directly resulting from the reaction of step B is completely evaporated and recondensed in liquid form thereby obtaining a purified reacted mixture. Any available technique can be used to evaporate the liquid reacted mixture, for example heating and a vacuum can be used individually or in combination to evaporate the mixture. A conventional evaporation equipment (e.g. a rotary evaporation equipment) may be used. Following the evaporation of the liquid reacted mixture, a solid residue is formed comprising the residual basic catalyst and salts obtained as by-products of the reaction which can be discarded or recycled.

The purified reacted mixture obtained in step D, differently from the reacted mixture obtained in step B, is pure enough to be distilled in a conventional distillation equipment. This is performed in step E of the process of the present invention.

### Water Extraction Method:

In the optional step F of the process of the present invention the liquid reacted mixture directly resulting from the reaction of step B is mixed with water and subject to agitation and or stirring so to extract in the water phase the water soluble impurities such as residues of the basic catalyst and other impurities and by-products. The relative amounts of water and reacted mixture to use in this step are from 1:15 to 15:1 by weight, preferably from 1:5 to 5:1, more preferably from 2:1 to 1:2. Agitation can be performed with any suitable technique used for extractions as known to the skilled person and a separatory funnel or similar equipment can be used separate the water phase from the phase containing the aprotic polar solvent and the hydrofluoroethers. The resulting phase containing the aprotic polar solvent and the hydrofluoroethers once separated from the water phase constitutes the purified reacted mixture which is pure enough to be subject to distillation in a conventional distillation equipment in step G of the process of the present invention.

In all the embodiments distillation allows to separate the hydrofluoroethers from the solvent. Distillation can be performed using conventional techniques and if necessary can be repeated to further purify the composition. In general the solvent will be recovered with known methods in order to be reused.

The Evaporation/recondensation method described above is in general preferred to the water extraction method because the water extraction method generates a large amount of waste water which is contaminated with the impurities of the system and therefore needs to be treated before being discarded or reused.

Both methods lead to a composition according to the invention wherein the composition consists essentially of one or more hydrofluoroether according to formula (I) and one or more hydrofluoroether of formula (II) in the weight ratio which is required by the invention.

The process of the invention can be carried out under mild conditions, additionally and a very high yield of hydrofluoroethers is obtained.

Should the disclosure of any patents, patent applications, and publications which are incorporated herein by reference conflict with the description of the present application to the extent that it may render a term unclear, the present description shall take precedence.

The invention will be now described in more detail with reference to the following examples, whose purpose is merely illustrative and not intended to limit the scope of the invention. Should the disclosure of any patents, patent applications, and publications which are incorporated herein by reference conflict with the description of the present application to the extent that it may render a term unclear, the present description shall take precedence.

### EXAMPLES

### Analysis

Product identification were performed by NMR (F-NMR and H-NMR) and GC and GC-MS analysis (GC using CP-WAX52CB column and CP-Sil8CB column for GC-MS peaks attribution).

### Example 1—Ethylene Glycol+TFE+Evaporation

37 g of ethylene glycol, 211 g of acetonitrile and 9.4 g of sodium hydroxide were loaded into a 600 ml stirred Hastelloy reactor. After purging with nitrogen and vacuum at 0.3 bar, the reactor was heated at 50° C. and, under stirring, pressurized up to 11 bar with TFE (tetrafluoroethylene).

After 6 hours stirring was stopped, reactor was cooled and after purging residues of TFE with nitrogen, a reacted mixture was recovered rinsing with 106 g of additional acetonitrile and discharged.

The collected reacted mixture (480 g) was then transferred in a glass flask and evaporated under vacuum at 1 mbar in a rotary evaporator heating the flask at 90° C. Solid by-products (14.5 g) were discarded and a clear colorless purified reacted mixture (445 g) was collected.

The purified reacted mixture was then distilled in a glass distillation equipment with a flask, a packed column and a condenser, equipped with vacuum pump. Distillation was carried out increasing temperature from 100° C. to 155° C. and with reduced pressure from 950 mbar down to 50 mbar. The distilled product contained 142 g of diether HCF₂CF₂—O—CH₂CH₂—O—CF₂CF₂H and 0.02 g of monoether HCF₂CF₂—O—CH₂CH₂—OH (0.01% by weight of the diether). Yield of the overall process based on the load of ethylene glycol was 90.3%.

### Example 2—Ethylene Glycol+TFE+Water Extraction

7231 g of acetonitrile, 2200 g of ethylene glycol and 1290 g of sodium hydroxide were loaded into a 22 L stirred Hastelloy reactor. After purging 4 times with nitrogen and vacuum at 0.3 bar, the reactor was heated at 60° C. and, under stirring, pressurized up to 3.5 bar with TFE (tetrafluoroethylene) and TFE was continuously fed maintain the initial pressure. During the reaction the reactor was cooled since the reaction was vigorous and exothermic. About 1300 g/h of TFE were consumed. After 7.5 hours the reaction stopped and TFE supply was suspended. The reactor was left at 60° C. and stirring and cooled to 25° C., excess TFE was purged with nitrogen, a reacted mixture was recovered and discharged.

The collected reacted mixture was then transferred in 200 L vessel and washed with 150 L demineralized water stirring at 25° C. for 6 hours and left to separate for 10 hours. The bottom organic layer was then separated (9300 g) and batch distilled in a rectification column (length 2.70 m, internal diameter 40 mm, filled with packing ¼″ Teflon® cylinders, and a 10 L bottom reboiler).

Distillation of the hydrofluoroethers was carried out under reduced pressure ca. 80 mbarA at 81° C. (head reflux ratio 10:1).

The hydrofluoroether mixture obtained was 8060 g, and was found to contain 8052 g of diether HCF2CF2-O—CH2CH2-O—CF2CF2H and 8 g of monoether HCF2CF2-O—CH2CH2-OH (0.1% by weight of the diether). Yield of the overall process based on the load of ethylene glycol was 86%.

When comparing the purity and the yield of the process of the present invention with that of the prior art it is clear that the process of the present invention provides for higher yields, higher purity of the hydrofluoroethers obtained.

Possible modifications and/or additions may be made by those skilled in the art to the hereinabove disclosed and illustrated embodiment while remaining within the scope of the following claims.

1. A liquid composition comprising one or more compound of formula (I): 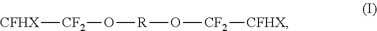
one or more compound of formula (II): 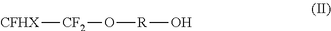
wherein:
R in formulas (I) and (II) is independently selected from C2-C10 divalent linear or branched alkyl, optionally including a cycle, an aromatic ring and/or oxygen heteroatoms engaged in ether bonds,
X is selected from halogens, H, Rf, wherein Rf is selected from:
a C1-C8 fully or partially fluorinated linear or branched alkyl optionally including a cycle, or
a C1-C3 fully or partially fluorinated alkoxy,
the two instances of X in formula (I) being the same or different,
the amount of the one or more compounds of formula (II) is from 0.002 to 5% by weight of the one or more compounds of formula (I).

2. The composition according to claim 1 wherein R is selected from:

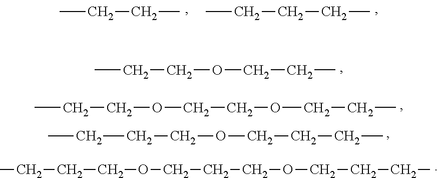

3. The composition according to claim 1, wherein X is selected from halogen, H and Rf, Rr being selected from a C1-C8 fully or partially fluorinated linear or branched alkyl optionally including a cycle, or a C1-C3 fully or partially fluorinated alkoxy.

4. The composition according to any preceding claim 1 wherein R is —CH₂—CH₂— and X is F.

5. The composition according to claim 1 wherein the the amount of the one or more compounds of formula (II) is from 0.005 to 3%, by weight of the total amount of said one or more compounds of formula (I).

6. The composition according to claim 1 wherein the the total amount of said compounds according to formulas (I) and (II) is at least 50%, based on the total weight of the composition.

7. A process for making the composition of claim 2, the process comprising:
A) providing a mixture comprising one more polar aprotic organic solvents having a boiling point measured at atmospheric pressure of from of 60 to 170° C., and one or more bifunctional alcohol having general formula:
HO—R—OH (III)
wherein R— is selected from C2-C10 divalent linear or branched alkyl, optionally including a cycle, an aromatic ring and/or O heteroatoms engaged in ether bonds,
B): reacting said and one or more bivalent alcohol with one or more fluorinated olefin having general formula
CFX═CF₂ (IV)
in the presence of a basic catalyst wherein
X is selected from halogens, H, Rf, wherein Rf is selected from:
a C1-C8 fully or partially fluorinated linear or branched alkyl optionally including a cycle, or
a C1-C3 fully or partially fluorinated alkoxy,
thus providing a reacted mixture comprising one or more hydrofluoroethers.

8. The process according to claim 7 wherein said fluorinated olefin is TFE.

9. The process according to claim 7 wherein said one or more compounds of formula (III) is selected from bivalent alcohols.

10. The process according to claim 7 said one or more polar aprotic organic solvent is acetonitrile.

11. The process according to claim 7 comprising an additional step C wherein said hydrofluoroethers are directly extracted from said reacted mixture.

12. The process according to claim 7 wherein said process further comprises the steps of
D) evaporating completely said reacted mixture and re-condense it in liquid form as a purified reacted mixture
E) separate said hydrofluoroethers in purified form from said purified reacted mixture via distillation.

13. The process according to claim 7, wherein said process further comprises the steps of
F) mix said reacted mixture with water, agitate the mixture thereby extracting water soluble impurities from said reacted mixture, and separate the purified reacted mixture as a water immiscible phase.
G) separate hydrofluoroethers in purified form from said purified reacted mixture via distillation.

14. The process of claim 9, wherein the bivalent alcohol is ethylene glycol.
